(19) United States
(12) Patent Application Publication
(10) Pub. No.: US 2004/0254627 A1
(43) Pub. Date: Dec. 16, 2004

(54) Stent with end adapted for flaring

(76) Inventors: Paul J. Thompson, New Hope, MN (US); Roy K. Greenberg, Cleveland, OH (US)

Correspondence Address: Merchant & Gould P.C., P.O. Box 2903, Minneapolis, MN, 55402-0903, US

(21) Appl. No.: 10/816,784

(22) Filed: Apr. 2, 2004

Related U.S. Application Data
(60) Provisional application No. 60/460,536, filed on Apr. 4, 2003.

(51) Int. Cl. A61F 2/06

(52) U.S. Cl. 623/1.11

(57) ABSTRACT

A stent for use in treating vascular diseases disclosed herein. The stent includes a main body and an end that can be flared relative to the main body. A predefined bend location is defined between the main body and the flared end.

### CROSS REFERENCE TO RELATED APPLICATIONS

The present application claims the benefit of U.S. Provisional Patent Application Ser. No. 60/460,536, filed Apr. 4, 2003, which application is incorporated herein by reference in its entirety.

### BACKGROUND OF THE INVENTION

1. Field of Invention

The present invention relates generally to luminal implants. More particularly, the present invention relates to stents for use in treating vascular disease.

2. Description of the Prior Art

Stents are widely used for supporting a lumen structure in a patient's body. For example, stents may be used to maintain patency of a coronary artery, other blood vessels or other body lumen.

Stents are commonly metal, tubular structures. Stents are passed through a body lumen in a collapsed state. At the point of an obstruction or other deployment site in the body lumen, the stent is expanded to an expanded diameter to support the lumen at the deployment site. Common structures for stents include coil structures and open cell tube structures. Example stents having open cell structures are disclosed in U.S. Pat. Nos. 6,358,274, 6,132,461 and 6,132,460, which are hereby incorporated by reference. Example stents having coil structures are disclosed in U.S. Pat. Nos. 4,768,507, 5,147,370, 5,372,600 and 5,246,445, which are hereby incorporated by reference.

In certain designs, stents are expanded by inflatable balloons at the deployment site. This type of stent is often referred to as a “balloon expandable” stent. Balloon expandable stents typically are configured to inelastically deform during expansion. Balloon expandable stents are frequently made of a material such as stainless steel. Other stents are so-called “self-expanding” stents. Self-expanding stents do not use balloons or other structures to expand the stents. An example of a self-expanding stent is a tube made of an elastically deformable material (e.g., a superelastic material such a nitinol). This type of stent is secured to a stent delivery device under tension in a collapsed state. At the deployment site, the stent is released so that internal tension within the stent causes the stent to self-expand to its enlarged diameter. Other self-expanding stents are made of so-called shape-memory metals. Such shape-memory stents experience a phase change at the elevated temperature of the human body. The phase change results in expansion from a collapsed state to an enlarged state.

Stents are commonly delivered percutaneously through the use of a catheter. Typically, a collapsed stent is mounted on a distal end of the catheter. While in the collapsed state, the stent is delivered to a deployment cite (e.g., a stenosis or blockage in a vessel such as an artery) by the catheter. Once delivered to the deployment cite, the stent is deployed to provide reinforcement for holding the vessel open. In the case of a balloon expandable stent, the stent is deployed by inflating a balloon positioned within the stent to cause the stent to inelastically expand. In the case of a self-expanding stent, the stent is commonly deployed by retracting a sheath to release the stent and allow the stent to self-expand.

Stents designed for implantation at different anatomical locations can have different physical characteristics. For example, stents for use in straight vessel sections generally have a straight, tubular configuration and stents for use in bifurcated vessels generally have bifurcated configurations. Stents have also been designed with flared ends for use at junctions between two vessels (i.e., at an ostium). Example flared stents are disclosed in U.S. Pat. Nos. 6,096,071; 5,868,777; 5,607,444; and 5,064,435.

### SUMMARY

One embodiment of the present disclosure relates to a stent having predefined bend locations that facilitate flaring the end of the stent.

A variety of advantages of the invention will be set forth in part in the description that follows, and in part will be apparent from the description, or may be learned by practicing the invention. It is to be understood that both the foregoing general description and the following detailed description are exemplary and explanatory only and are not restrictive of the invention as claimed.

### BRIEF DESCRIPTION OF THE DRAWINGS

FIG. 1 is a perspective view of an embodiment of a stent having features that are examples of inventive aspects in accordance with the principles of the present disclosure;

FIG. 2 is a plan view of the interior of the stent of FIG. 1 with the stent having been cut longitudinally and laid flat;

FIG. 3 is a cross-sectional view taken along section line 3-3 of FIG. 2;

FIG. 4 is a cross-sectional view of an alternative predefined bend location in accordance with the principles of the present disclosure;

FIG. 5 is a cross-sectional view of still another predefined bend location in accordance with the principles of the present disclosure;

FIG. 6 is a cross-sectional view of a further predefined bend location in accordance with the principles of the present disclosure;

FIG. 7 is a cross-sectional view taken along section line 7-7 of FIG. 3;

FIG. 8 is a cross-sectional view taken along section line 8-8 of FIG. 3;

FIG. 9 shows the stent of FIG. 1 deployed at the junction between the aorta and a renal artery;

FIG. 10 shows the stent of FIG. 1 being used to secure a fenestrated graft within the aorta;

FIG. 11 is a plan view of an alternative stent having features that are examples of inventive aspects in accordance with the principles of the present disclosure;

FIG. 12 is a plan view of a further stent having features that are examples of inventive aspects in accordance with the principles of the present disclosure; and

FIG. 13 shows still another stent having features that are examples of inventive aspects in accordance with the principles of the present disclosure.

### DETAILED DESCRIPTION

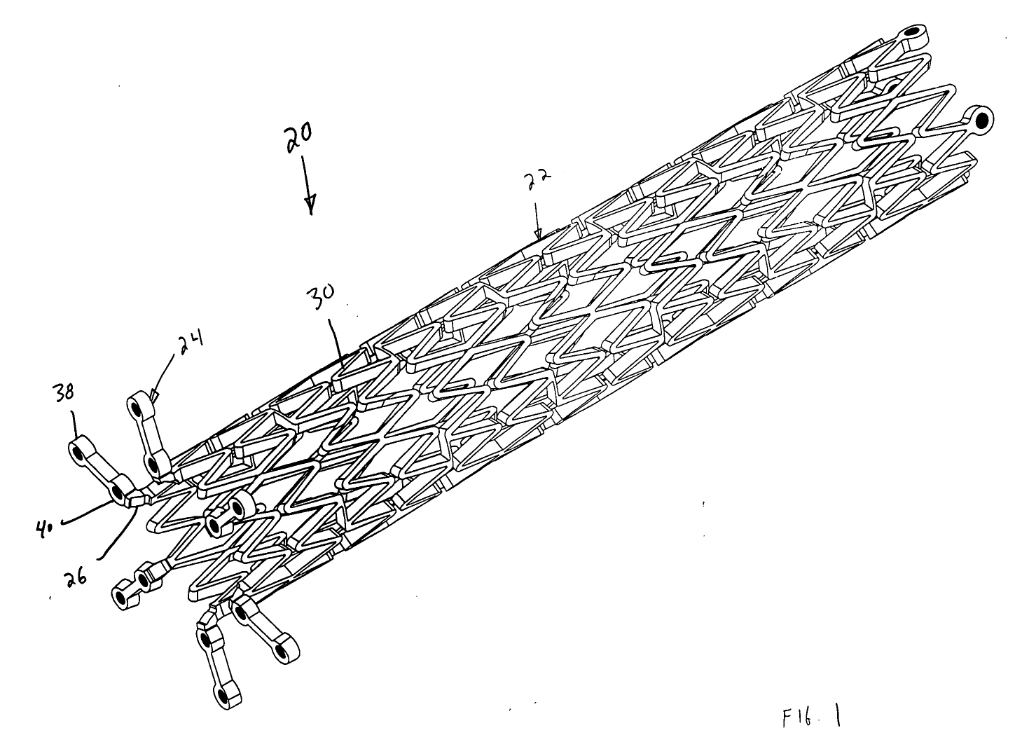

FIG. 1 illustrates a stent 20 having features that are examples of inventive concepts in accordance with the principles of the present disclosure. The stent is movable between a radially collapsed orientation (not shown) and an expanded/deployed orientation (shown in FIG. 1). The stent 20 includes a main body 22 and an end 24 adapted to be flared relative to the main body 22. Predefined bend locations such as notches 26 are located between the main body 22 and the end 24. The notches 26 facilitate flaring the end 24 relative to the main body 22 and reduce the likelihood that portions of the end 24 could fracture from the main body 22.

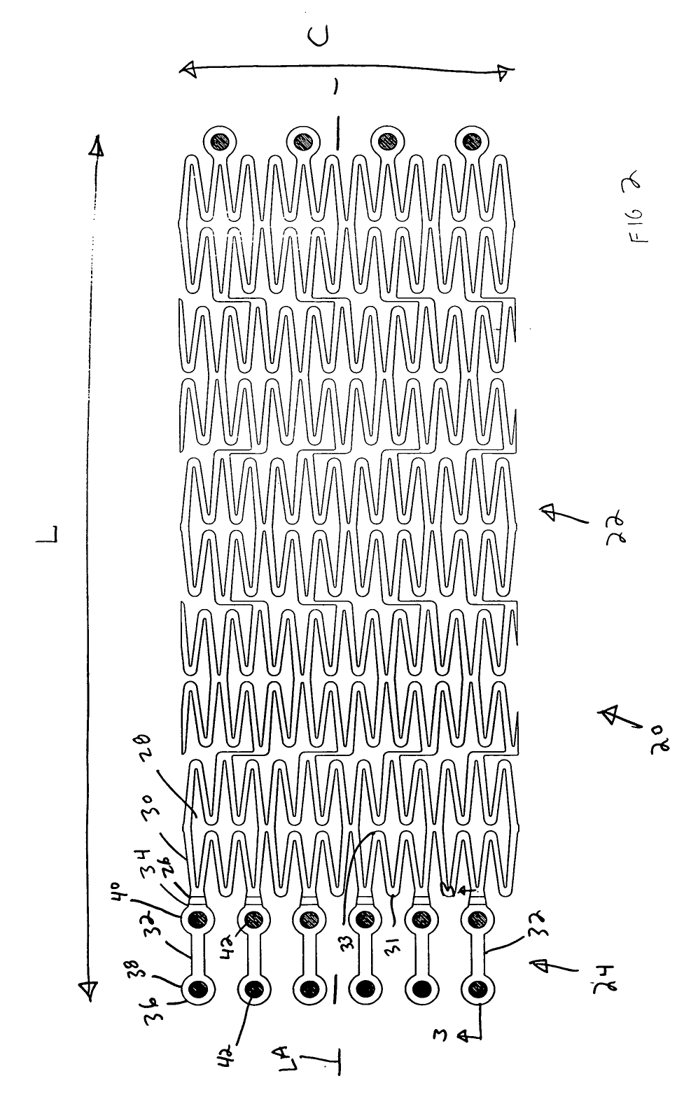

Referring to FIG. 2, the main body 22 of the stent 20 has a lattice or reticulated configuration and defines a plurality of open cells 28. The open cells 28 extend through the main body 22 from an exterior to an interior of the main body 22. The cells 28 are defined by support members 30 (i.e., struts). The support members 30 define an undulating pattern having a plurality of peaks 31 and valleys 33. The stent 20 has a length L that extends along a longitudinal axis LA of the stent, and a circumference C. The undulating pattern defined by the support members 30 extends around the circumference C of the stent.

The end 24 of the stent 20 is defined by a plurality of struts such as cantilever members 32 that project outwardly from the main body 22. As shown in FIG. 2, the members 32 are parallel to the longitudinal axis LA prior to expansion. Each of the cantilever members 32 has a base end 34 and a free end 36. The base ends 34 are integrally connected to the main body 22 adjacent the notches 26. In a preferred embodiment, the cantilever members 32 and the support members 30 are made by cutting material from a single tube of material. Therefore, in a preferred embodiment, the cantilever members 32 and the main body 22 are unitarily/monolithically connected.

Referring to FIG. 2, each of the cantilever members 32 includes first and second enlargements 38 and 40. The first enlargements 38 are located at the free ends 36 or tips of the cantilever members 32, while the second enlargements 40 are located adjacent the base ends 34 of the cantilever members 32. Inserts 42 made of a material visible under x-ray are positioned within the enlargements 38, 40. In one embodiment, the inserts 32 include rivets made of tantalum. Other material such as gold, platinum, tungsten, iridium and niobium could also be used.

Referring to FIGS. 2 and 3, the notches 26 are located between the cantilever members 32 and the main body 22 of the stent 20. As shown in FIG. 3, the depicted notch 26 is defined at an interior surface 44 of the stent 20. Still referring to FIG. 3, the notch 26 includes two parallel surfaces 46 interconnected by a curved surface 48. However, it will be appreciated that other notch configurations such as rectangular, semi-circular, triangular, elliptical or other shapes could also be used.

It will be appreciated that the notch 26 provides a relief for facilitating bending the cantilever members 32. The relief includes a reduced wall thickness W_{T1} at the notch 26 as compared to a wall thickness W_{T2} at the main body 22 immediately adjacent to the notch. In one embodiment, the wall thickness W_{T2} is in the range of 0.004-0.015 inches, and the wall thickness W_{T1} is smaller than the wall thickness W_{T2} by an amount in the range of 0.0005-0.010 inches, or 0.0005-0.005 inches, or 0.001-0.005 inches. In one embodiment, the wall thickness W_{T2} is about 0.005-0.009 inches and the thickness W_{T1} is smaller by about 0.001-0.003 inches. Of course, the above dimensions are merely examples and embodiments of the present invention can include dimensions other than those specifically listed above.

Referring to FIGS. 7 and 8, because of the difference between the wall thicknesses W_{T1} and W_{T2,} the cross-sectional area of the wall of the stent 20 is smaller adjacent the notch 26 as compared to at the main body 22. In one embodiment, the cross-sectional area of the wall at the notch 26 is in the range of 5-80% smaller than the cross-sectional area of the main body wall at a location immediately adjacent to the predefined bend location 26. In other embodiments, the cross-sectional area of the wall at the notch 26 is in the range of 15-60%, 20-50%, 20-40% or 20-30% smaller than the cross-sectional area of the main body immediately adjacent the notch. Of course, the above percentages are merely examples and embodiments of the present invention can include cross-sectional variations other than those specifically listed above.

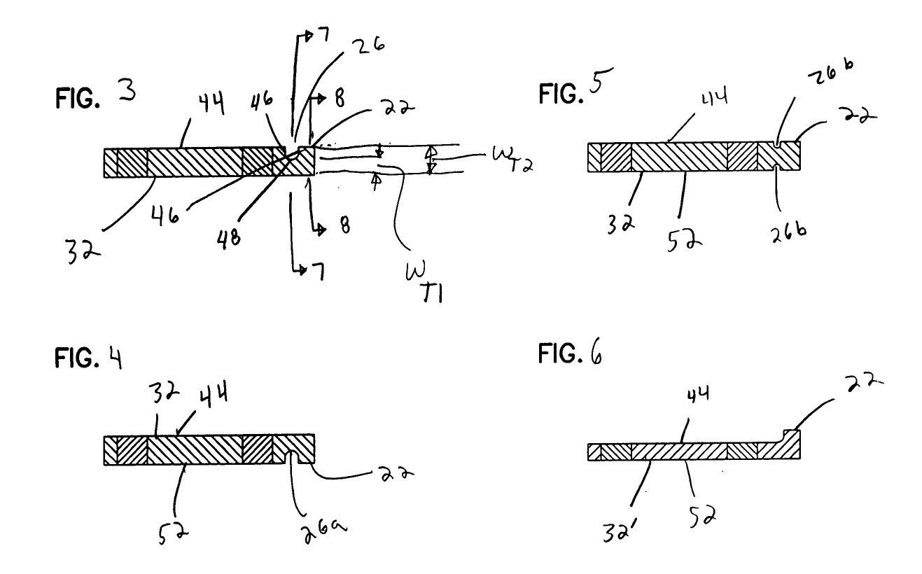

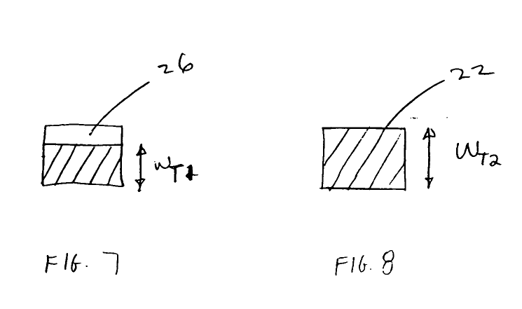

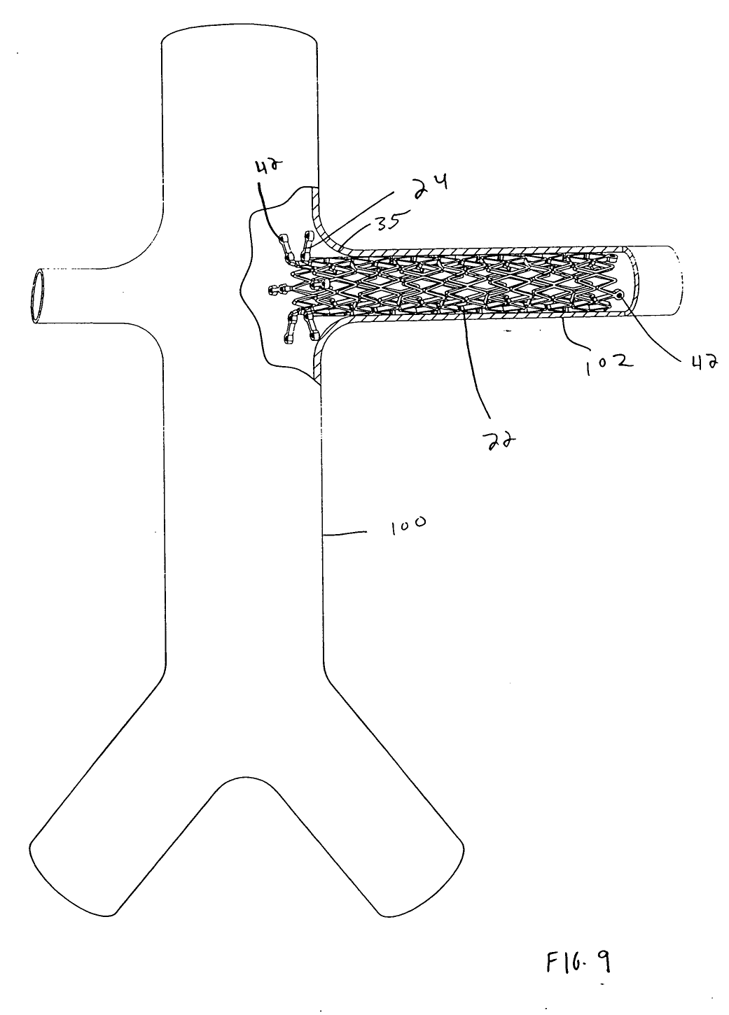

FIG. 4 illustrates an alternative predefined bend location defined by a notch 26a located at an exterior surface 52 of the stent 20. FIG. 5 illustrates still another predefined bend location defined by notches 26b located at interior and exterior surfaces 44 and 52 of the stent 20. FIG. 6 illustrates a predefined bend location defined by a shoulder 26c that provides a relief. As shown in FIG. 6, the shoulder 26c reduces the wall thickness of the entire cantilever arm 32′ relative to the wall thickness of the main body 22 of the stent.

It will be appreciated that the various aspects of the present disclosure are applicable to balloon expandable and self-expanding stents. Materials for making balloon expandable stents include stainless steel, MP35N and elgiloy. Materials for making self-expanding stents include nitinol and elgiloy.

To manufacture a balloon expandable embodiment of the present invention, the main body 22 and the end 24 can be cut (e.g., laser cut or photo etched) from a tube of material such as stainless steel. Preferably, the tube is cut while at a diameter corresponding to a deployed diameter of the stent. During the cutting process, the material corresponding to the cells 28 is removed while the support members 30 are left uncut. Similarly, the material corresponding to the regions between the cantilever members 32 is removed leaving the members 32 uncut. During this process, the notches 26 are also cut into the body of the stent. After the cutting process, the inserts 42 can be placed in the enlarged portions 38 and 40 of the cantilever members 32.

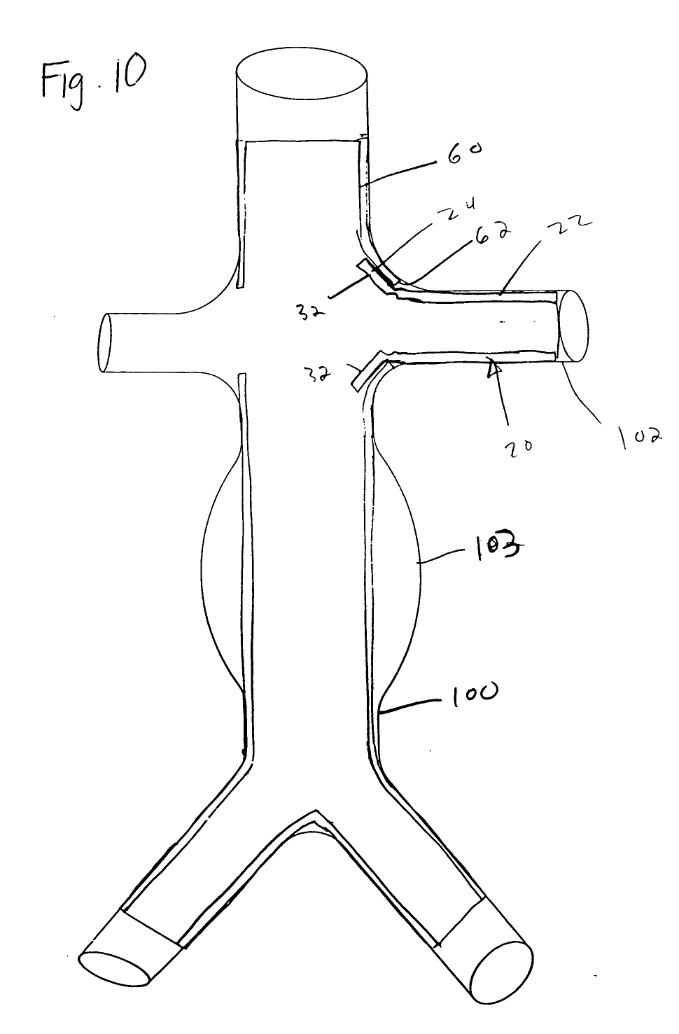

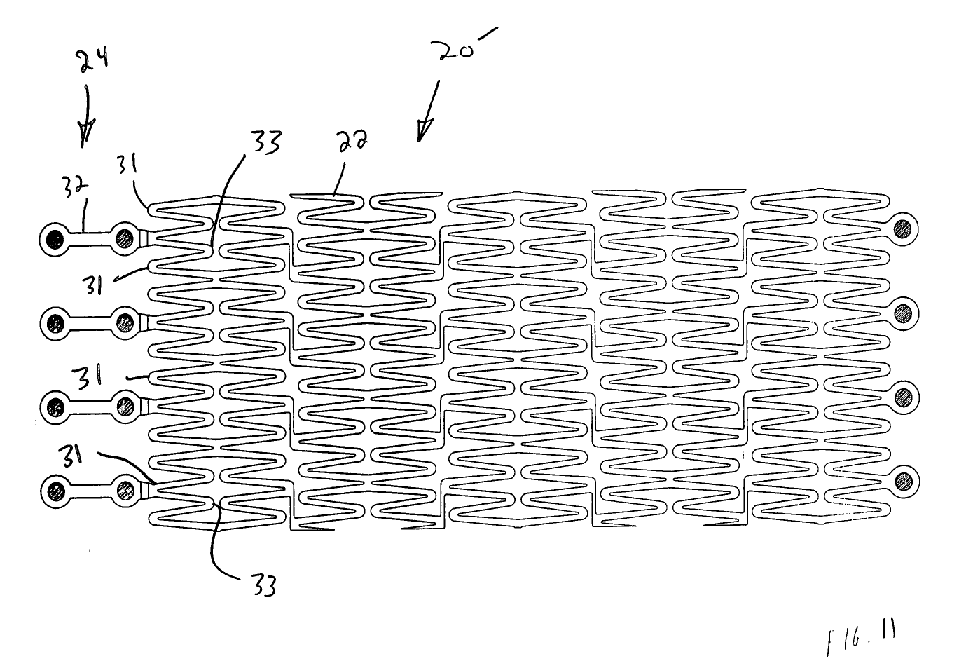

In the case of a balloon expandable stent, the stent is preferably deployed via a balloon catheter. The stent is deployed by guiding the catheter through a patient's vasculature until the stent is located at the desired deployment site. For example, FIG. 9 shows a deployment site located at a junction between an aorta 100 and a renal artery 102. Once the stent 20 is positioned at the appropriate site, the stent is deployed by expanding the balloon. In one embodiment, the balloon may initially be used to expand the main body 22 of the stent. After the main body has been expanded, the balloon can be moved so that the balloon is located only within the region defined by the end 24. The balloon can then be further inflated to flare the cantilever members 32 outwardly at the ostium 35 of the junction between the renal artery 102 and the aorta 100 (see FIG. 9). The stent 20 can also be used to treat an aortic aneurysm (e.g., an abdominal aortic aneurysm 103) by securing a fenestrated graft 60 within the aorta 100. FIG. 10 shows stent 20 projecting through an opening 62 in the graft 60. The flared end 24 is expanded to trap a portion of the graft 60 against the ostium 35. A stent could similarly be used at the other renal artery 102.

A self-expanding embodiment of the stent 20 is preferably made by cutting a tube of super elastic material (e.g., nitinol) so as to define the support members 30 and the cantilever members 32 as previously described. Exemplary cutting methods include laser cutting, photo etching or electric discharge machining. After the stent 20 has been cut, the inserts 42 are secured to the stent 20 and the deployed/expanded shape of the stent 20 (shown in FIGS. 1 and 9) is set. Preferably the shape is set by a temperature shape setting process as is conventionally used with shape-memory/superelastic devices.

In the case of a self-expanding stent, the stent can be implanted at a junction such as the junction between the aorta 100 and the renal artery 102 (see FIG. 9) through the use of a catheter having a retractable sheath. The stent is manipulated to the deployment site while in a compressed orientation. Once positioned at the deployment site, the sheath can be retracted thereby allowing the stent to self expand to the configuration shown in FIG. 9. The x-ray visible inserts 42 assist in determining whether the stent 20 has been properly positioned.

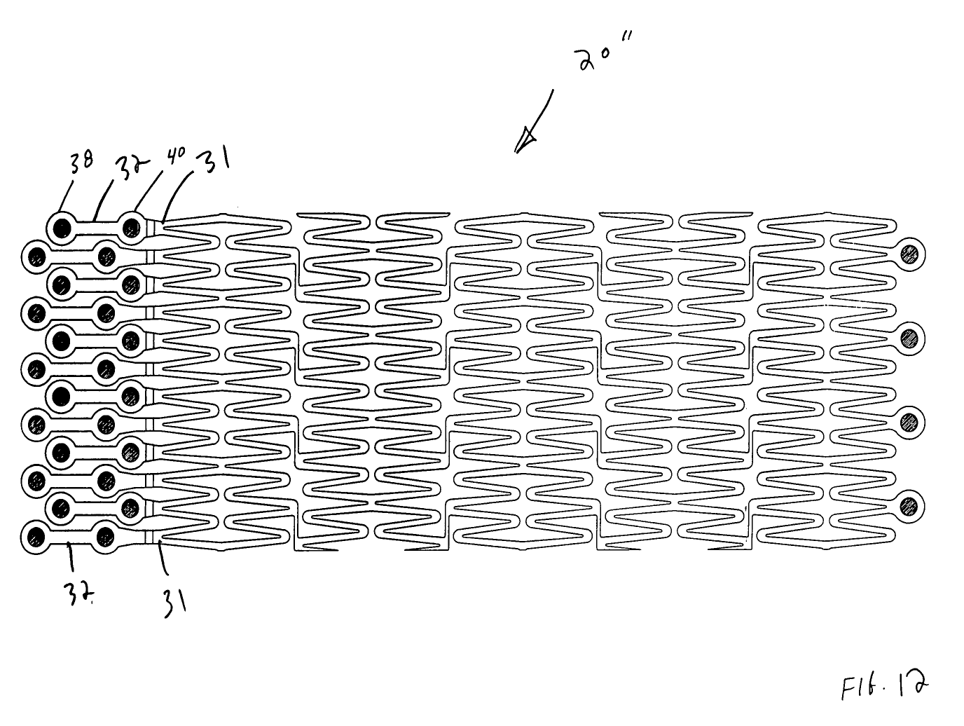

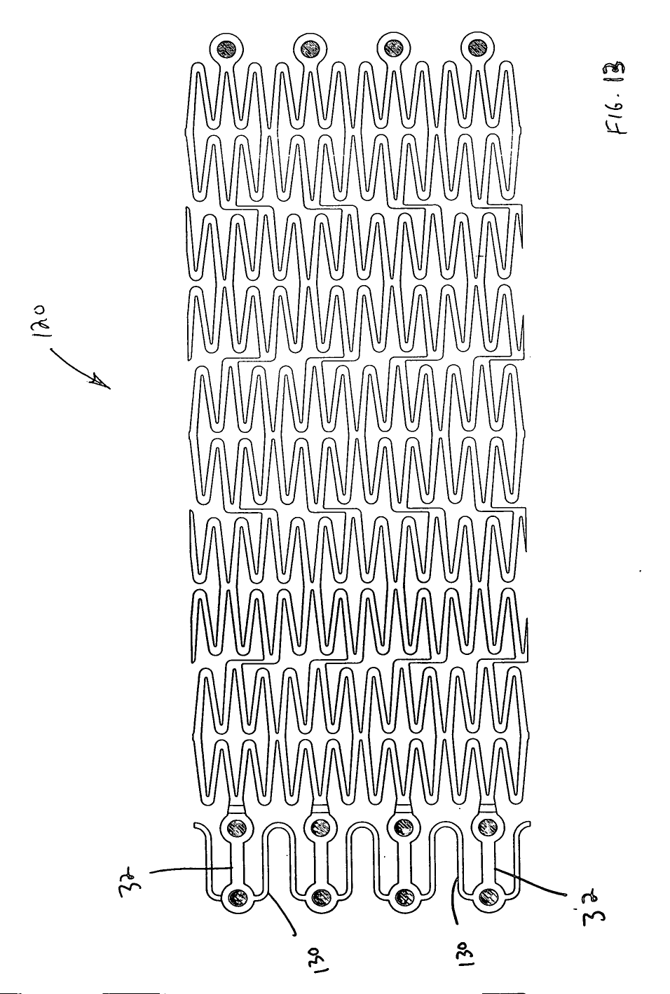

In the embodiment of FIGS. 1 and 2, the cantilever members 32 are connected to every other peak 31 of the main body. FIG. 11 shows an alternative stent 20′ where cantilever members 32 are connected to every third peak 31. FIG. 12 shows a further embodiment of a stent 20″ where cantilever members 32 are connected to every peak 31. In this embodiment, the enlargements 38, 40 of adjacent cantilevers are axially/longitudinally offset from one another to provide clearance.

FIG. 13 shows another stent 120 having features that are examples of inventive aspects in accordance with the principles of the present disclosure. The stent includes linking members 130 that extend between cantilever members 32. The linking members 130 are configured to straighten as the cantilever members 32 are flared.

It has been shown how the objects of the invention have been attained in a preferred manner. While a preferred use is at the ostiums between the aorta and the renal arteries, it will be appreciated that stents in accordance with the present disclosure could be used at any other junction between two vessels or for any other application suitable for a flared stent. Modifications and equivalents of the disclosed concepts are intended to be included within the scope of the claims.

What is claimed is:

1. A stent comprising:
a main body defining a plurality of cells, the main body including opposite ends;
an end structure adapted to be flared relative to the main body, the end structure being located adjacent at least one of the ends of the main body; and
the end structure including predefined bend locations for facilitating flaring the end structure relative to the main body.

2. The stent of claim 1, wherein the end structure includes a plurality of cantilever members having base ends connected to the main body at the predefined bend locations.

3. The stent of claim 1, wherein each predefined bend location includes one or more notches.

4. The stent of claim 1, wherein the predefined bend locations include areas of reduced cross-section as compared to areas of adjacent locations.

5. The stent of claim 4, wherein the areas of reduced cross-section are in the range of 15-60 percent smaller than the areas of the adjacent locations.

6. The stent of claim 1, wherein the predefined bend locations include notches provided at interior and exterior surfaces of the stent.

7. The stent of claim 1, wherein the predefined bend locations include notches provided at exterior surfaces of the stent.

8. The stent of claim 1, wherein the predefined bend locations include notches provided at interior surfaces of the stent.

9. The stent of claim 2, wherein the cantilever members include enlargements in which x-ray visible markers are positioned.

10. The stent of claim 1, wherein the predefined bend locations include shoulders.

11. The stent of claim 1, wherein the end structure includes a plurality of end struts having base ends connected to the main body.

12. The stent of claim 11, further comprising linking members that extend between the end struts.

13. The stent of claim 12, wherein the linking members are configured to straighten as the end struts are flared.

14. A stent comprising:
a main body defining a plurality of cells, the main body having opposite ends;
a plurality of end struts adapted to be flared relative to the main body, the end struts being integrally connected with at least one of the ends of the main body; and
the end struts including regions of reduced radial wall thickness for facilitating flaring the end struts relative to the main body.

15. The stent of claim 14, wherein the end struts are connected to the main body at connection locations, and wherein the regions of reduced radial wall thickness are located adjacent to the connection locations.

16. The stent of claim 14, wherein the regions of reduced radial wall thickness are provided by notches.

17. The stent of clam 14, wherein the regions of reduced radial wall thickness are defined by shoulders.

18. A stent comprising:
a main body including a plurality of support members defining a plurality of open cells, the support members extending about a circumference of the main body and each defining an undulating pattern having a plurality of peaks and valleys;
a plurality of end struts adapted to be flared relative to the main body, the end struts being connected to at least some of the peaks of the main body; and
the end struts defining notches for facilitating flaring the end struts relative to the main body.

19. The stent of claim 18, wherein the main body includes an end support member having a plurality of peaks and valleys, and wherein the end struts are connected to every other peak of the end support member.

20. The stent of claim 18, wherein the main body includes an end support member having a plurality of peaks and valleys, and wherein the end struts are connected to every third peak of the end support member.

21. The stent of claim 18, wherein the main body includes an end support member having a plurality of peaks and valleys, and wherein the end struts are connected to every peak of the end support member.

22. The stent of claim 18, wherein each end strut includes two enlargements including radiopaque markers.

23. A method for implanting a stent at a junction between first and second vessels, the stent including a main body and an end structure adapted to be flared relative to the main body, the stent also including predefined bend locations for facilitating flaring the end structure relative to the main body, the method comprising:
positioning the stent such that the main body is located within the first vessel, the end structure extends into the second vessel, and the predefined bend locations are located adjacent the junction between the first and second vessels;
radially expanding the main body into contact with an interior surface of the first vessel; and
flaring the end structure relative to the main body such that the end portion generally conforms with an interior surface of the second vessel, the end structure being flared by bending the stent at the predefined bend locations while maintaining the predefined bend locations adjacent the junction between the first and second vessels.

24. A stent comprising:
a main body defining a plurality of cells, the main body having opposite ends;
a plurality of end struts adapted to be flared relative to the main body; and
the end struts having lengths, and the end struts being thinned along their lengths relative to the main body for facilitating flaring the end struts relative to the main body.
